(19) United States
(12) Patent Application Publication
(10) Pub. No.: US 2026/0240796 A1
(43) Pub. Date: Aug. 20, 2026

(54) COSMETIC COMPOSITION COMPRISING A FATTY ACID AND A HYDROXY CARBOXYLIC ACID AND ITS USE IN THE TREATMENT OF ACNE

(71) Applicant: Conopco, Inc., d/b/a UNILEVER, Hoboken, NJ (US)

(72) Inventors: Haijun HONG, Shanghai (CN); Lin LI, Shanghai (CN); Zhengrong LI, Shanghai (CN); Shuqi YANG, Shanghai (CN)

(73) Assignee: Conopco, Inc., d/b/a UNILEVER, Hoboken, NJ (US)

(21) Appl. No.: 19/161,424

(22) PCT Filed: Feb. 20, 2024
(86) PCT No.: PCT/EP2024/054209

§ 371 Date: Sep. 2, 2025

(30) Foreign Application Priority Data
Mar. 8, 2023 (WO) PCT/CN2023/080333
Apr. 26, 2023 (EP) 23170008.9

(51) Int. Cl. A61K 31/19; A61K 8/36; A61K 8/365; A61P 17/10; A61Q 17/00; A61Q 19/10

(52) U.S. Cl. CPC A61K 31/19; A61K 8/361; A61K 8/365; A61P 17/10; A61Q 17/005; A61Q 19/10; A61K 2800/596

(57) ABSTRACT

Disclosed is a cosmetic composition comprising component (a) selected from C₈ to C₁₅ fatty acid and salt thereof; and component (b) selected from C₂ to C₆ hydroxy carboxylic acid and salt thereof, wherein the weight ratio of total component (a) to total component (b) is at least 3:1, the fatty acid is C₁₀ to C₁₄ fatty acid and the hydroxy carboxylic acid is C₂ to C₄ alpha hydroxy carboxylic acid.

### FIELD OF THE INVENTION

The present invention relates to a cosmetic composition comprising component (a) selected from C₈ to C₁₅ fatty acid and salt thereof wherein the fatty acid is C₁₀ to C₁₄ fatty acid; and component (b) selected from C₂ to C₆ hydroxy carboxylic acid and salt thereof wherein the hydroxy carboxylic acid is C₂ to C₄ alpha hydroxy carboxylic acid with specific ratio. It was surprisingly found that such composition is unexpectedly capable of delivering a synergistic anti-acne and anti-microbial benefit.

### BACKGROUND OF THE INVENTION

Acne, also known as Acne vulgaris, is a common skin condition that affects nearly all adolescents and adults at some point in their lives. It has been observed that acne usually occurs in areas rich in sebaceous glands like the face, neck and back. A bacteria Propionibacterium acnes (P. acnes) has also been implicated in occurrence of acne. It resides in the sebaceous glands. P. acnes uses sebum and by-product from surrounding skin tissue as sources of energy and nutrients, leading to acne or acne vulgaris.

Although many ways have been developed for treating acne, there is still a need to improve the efficacy of anti-acne. Therefore, the present inventors have developed a cosmetic composition comprising component (a) selected from C₈ to C₁₅ fatty acid and salt thereof wherein the fatty acid is C₁₀ to C₁₄ fatty acid; and component (b) selected from C₂ to C₆ hydroxy carboxylic acid and salt thereof wherein the hydroxy carboxylic acid is C₂ to C₄ alpha hydroxy carboxylic acid with specific ratio. It was surprisingly found that such composition is unexpectedly capable of providing a synergistic anti-acne benefit.

### SUMMARY OF THE INVENTION

In a first aspect, the present invention is directed to a cosmetic composition comprising: component (a) selected from C₈ to C₁₅ fatty acid and salt thereof; and component (b) selected from C₂ to C₆ hydroxy carboxylic acid and salt thereof, wherein the weight ratio of total component (a) to total component (b) is at least 3:1, the fatty acid is C₁₀ to C₁₄ fatty acid and the hydroxy carboxylic acid is C₂ to C₄ alpha hydroxy carboxylic acid.

In a second aspect, the present invention is directed to use of a cosmetic composition of the present invention for providing anti-acne or anti-bacterial benefit.

In a third aspect, the present invention is directed to a cosmetic composition of the present invention for providing anti-acne or anti-bacterial benefit.

In a fourth aspect, the present invention is directed to use of a combination of component (a) selected from C₈ to C₁₅ fatty acid and salt thereof; and component (b) selected from C₂ to C₆ hydroxy carboxylic acid and salt thereof, wherein the weight ratio of total component (a) to total component (b) is at least 3:1, for use as anti-acne or anti-bacterial agent.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

Preferably, the C₈ to C₁₅ fatty acid comprises C₁₂ to C₁₄ fatty acid. The C₈ to C₁₅ fatty acid is C₁₀ to C₁₄ fatty acid and even more preferably C₁₂ to C₁₄ fatty acid. Preferably the fatty acid has a straight chain with length of 8 to 14, preferably 10 to 14 carbon atoms. The fatty acid is preferably saturated fatty acid. Preferably, the fatty acid is fatty acid without substitution. More preferably the fatty acid is saturated fatty acid without substitution, even more preferably C₁₀ to C₁₄ saturated fatty acid without substitution. Substitution refers to the hydrogen in the carbon chain was substituted by heteroatom. Heteroatom refers to any atom that is not carbon or hydrogen. Preferably, the fatty acid is lauric acid, myristic acid, or a combination thereof. More preferably, the fatty acid comprises lauric acid. Even more preferably, the fatty acid is lauric acid. Salt of C₈ to C₁₅ fatty acid is preferably potassium, sodium and/or ammonium salt of fatty acid, and more preferably sodium salt of fatty acid. Salt of the fatty acid is preferably potassium, sodium and/or ammonium salt of fatty acid, and more preferably sodium salt of fatty acid.

Preferably, component (a) is selected from lauric acid and salt of lauric acid. More preferably component (a) is lauric acid.

The hydroxy carboxylic acid is C₂ to C₄ alpha hydroxy carboxylic acid, preferably the C₂ to C₆ hydroxy carboxylic acid comprises lactic acid. Most preferably the C₂ to C₆ hydroxy carboxylic acid is lactic acid.

Salt of C₂ to C₄ hydroxy carboxylic acid is potassium, sodium and/or ammonium salt of C₂ to C₆ hydroxy carboxylic acid, and more preferably sodium salt of C₂ to C₆ hydroxy carboxylic acid. Salt of hydroxy carboxylic acid is potassium, sodium and/or ammonium salt of hydroxy carboxylic acid, and more preferably sodium salt of hydroxy carboxylic acid.

Preferably, component (b) is selected from lactic acid and salt of lactic acid. More preferably, component (b) is lactic acid.

Preferably, the weight ratio of total weight of C₈ to C₁₅ fatty acid and salt thereof to the total weight of C₂ to C₆ hydroxy carboxylic acid and salt thereof is 3:1 to 30:1, more preferably 3:1 to 15:1, even more preferably 3.5:1 to 10:1, still even more preferably 3.5:1 to 8.5:1 and most preferably 4:1 to 7:1.

Preferably, the component (a) is selected from C₁₂ to C₁₄ fatty acid and salt thereof, and component (b) is C₂ to C₆ hydroxy carboxylic acid or salt thereof. Preferably, the weight ratio of total weight of C₁₂ to C₁₄ fatty acid salt thereof to the total weight of C₂ to C₆ hydroxy carboxylic acid and salt thereof is 3:1 to 30:1, more preferably 3:1 to 15:1, even more preferably 3.5:1 to 10:1, still even more preferably 3.5:1 to 8.5:1 and most preferably 4:1 to 7:1.

Preferably, the component (a) is selected from C₁₂ to C₁₄ fatty acid and salt thereof, and component (b) is selected from C₂ to C₄ alpha hydroxy carboxylic acid and salt thereof. Preferably, the weight ratio of total weight of C₁₂ to C₁₄ fatty acid salt thereof to the total weight of C₂ to C₄ alpha hydroxy carboxylic acid and salt thereof is 3:1 to 30:1, more preferably 3:1 to 15:1, even more preferably 3.5:1 to 10:1, still even more preferably 3.5:1 to 8.5:1 and most preferably 4:1 to 7:1.

Preferably, the component (a) is selected from lauric acid, myristic acid, salt thereof, and combination thereof, and component (b) is selected from C₂ to C₄ alpha hydroxy carboxylic acid and salt thereof. Preferably, the weight ratio of total weight of lauric acid, myristic acid, and salt thereof to the total weight of C₂ to C₄ alpha hydroxy carboxylic acid and salt thereof is 3:1 to 30:1, more preferably 3:1 to 15:1, even more preferably 3.5:1 to 10:1, still even more preferably 3.5:1 to 8.5:1 and most preferably 4:1 to 7:1.

Preferably, the component (a) is selected from lauric acid, myristic acid, salt thereof, and combination thereof, and component (b) is selected from lactic acid and salt of lactic acid. Preferably, the weight ratio of total weight of lauric acid, myristic acid, and salt thereof to the total weight of lactic acid and salt of lactic acid is 3:1 to 30:1, more preferably 3:1 to 15:1, even more preferably 3.5:1 to 10:1, still even more preferably 3.5:1 to 8.5:1 and most preferably 4:1 to 7:1.

Preferably, the component (a) is lauric acid, and component (b) is lactic acid. Preferably, the weight ratio of lauric acid to lactic acid is 3:1 to 30:1, more preferably 3:1 to 15:1, even more preferably 3.5:1 to 10:1, still even more preferably 3.5:1 to 8.5:1 and most preferably 4:1 to 7:1.

Preferably, the total amount of C₈ to C₁₅ fatty acid and salt thereof is 0.001 to 8%, more preferably 0.05 to 5%, even more preferably 0.2 to 2% and most preferably 0.4 to 1.2% by weight of the composition.

Preferably, the total amount of component (a) is 0.001 to 8%, more preferably 0.05 to 5%, even more preferably 0.2 to 2% and most preferably 0.4 to 1.2% by weight of the composition.

Preferably, the total amount of C₁₀ to C₁₄ fatty acid and salt thereof is 0.001 to 8%, more preferably 0.05 to 5%, even more preferably 0.2 to 2% and most preferably 0.4 to 1.2% by weight of the composition. Preferably, the total amount of C₁₂ to C₁₄ fatty acid and salt thereof is 0.001 to 8%, more preferably 0.05 to 5%, even more preferably 0.2 to 2% and most preferably 0.4 to 1.2% by weight of the composition.

Preferably, the total amount of lauric acid, myristic acid, and salt thereof is 0.001 to 8%, more preferably 0.05 to 5%, even more preferably 0.2 to 2% and most preferably 0.4 to 1.2% by weight of the composition. Preferably, the total amount of lauric acid and salt thereof is 0.001 to 8%, more preferably 0.05 to 5%, even more preferably 0.2 to 2% and most preferably 0.4 to 1.2% by weight of the composition. Preferably, the amount of lauric acid is 0.001 to 8%, more preferably 0.05 to 5%, even more preferably 0.2 to 2% and most preferably 0.4 to 1.2% by weight of the composition.

In another preferred embodiments, the total amount of component (a) is 0.01 to 30%, more preferably 0.1 to 18%, even more preferably 0.2 to 15%, and still even more preferably 4 to 12% by weight of the composition. Preferably, the total amount of C₁₀ to C₁₄ fatty acid and salt thereof is 0.01 to 30%, more preferably 0.1 to 18%, even more preferably 0.2 to 15%, and still even more preferably 4 to 12% by weight of the composition. Preferably, the total amount of C₁₂ to C₁₄ fatty acid and salt thereof is 0.01 to 30%, more preferably 0.1 to 18%, even more preferably 0.2 to 15%, and still even more preferably 4 to 12% by weight of the composition. Preferably, the amount of the total C₁₀ to C₁₄ fatty acid salt is 0.01 to 30%, more preferably 0.1 to 18%, even more preferably 0.2 to 15%, and still even more preferably 4 to 12% by weight of the composition. Preferably, the total amount of lauric acid and salt thereof is 0.01 to 30%, more preferably 0.1 to 18%, even more preferably 0.2 to 15%, and still even more preferably 4 to 12% by weight of the composition.

Preferably, the total amount of component (b) is 0.001 to 6%, more preferably 0.02 to 2%, even more preferably 0.05 to 1%, still even more preferably 0.08 to 0.7% and most preferably 0.1 to 0.4% by weight of the composition. Preferably, the amount of C₂ to C₄ alpha hydroxy carboxylic acid and salt thereof is 0.001 to 6%, more preferably 0.02 to 2%, even more preferably 0.05 to 1%, still even more preferably 0.08 to 0.7% and most preferably 0.1 to 0.4% by weight of the composition.

Preferably, the total amount of lactic acid and salt of lactic acid is 0.001 to 6%, more preferably 0.02 to 2%, even more preferably 0.05 to 1%, still even more preferably 0.08 to 0.7% and most preferably 0.1 to 0.4% by weight of the composition. Preferably, the amount of lactic acid is 0.001 to 6%, more preferably 0.02 to 2%, even more preferably 0.05 to 1%, still even more preferably 0.08 to 0.7% and most preferably 0.1 to 0.4% by weight of the composition.

The present invention provides a cosmetic composition comprising component (a) selected from C₁₀ to C₁₄ fatty acid fatty acid and salt thereof; component (b) selected from C₂ to C₄ alpha hydroxy carboxylic acid, wherein the weight ratio of total component (a) to total component (b) is at least 3:1. Preferably, the weight ratio of total weight of C₁₀ to C₁₄ fatty acid and salt thereof to the total weight of C₂ to C₄ alpha hydroxy carboxylic acid and salt thereof is 3:1 to 30:1, more preferably 3:1 to 15:1, even more preferably 3.5:1 to 10:1, still even more preferably 3.5:1 to 8.5:1 and most preferably 4:1 to 7:1.

The composition preferably comprises a cleansing surfactant. More than one cleansing surfactant may be included in the composition. The cleaning surfactant may be chosen from soap, non-soap anionic, cationic, non-ionic, amphoteric surfactant and mixtures thereof. Many suitable surface-active compounds are available and are fully described in the literature, for example, in “Surface-Active Agents and Detergents”, Volumes I and II, by Schwartz, Perry and Berch. The preferred surface-active compounds that can be used are soaps, non-soap anionic, non-ionic surfactant, amphoteric surfactant or a mixture thereof.

Suitable non-soap anionic surfactants include linear alkylbenzene sulphonate, primary and secondary alkyl sulphates, particularly C₈ to C₁₅ primary alkyl sulphates; alkyl ether sulphates; olefin sulphonates; alkyl xylene sulphonates; dialkyl sulphosuccinates; fatty acid ester sulphonates; or a mixture thereof. Sodium salts are generally preferred.

Most preferred non-soap anionic surfactant are linear alkylbenzene sulphonate, particularly linear alkylbenzene sulphonates having an alkyl chain length of from C₈ to C₁₅. It is preferred if the level of linear alkylbenzene sulphonate is from 0 wt % to 30 wt %, more preferably from 1 wt % to 25 wt %, most preferably from 2 wt % to 15 wt %, by weight of the total composition.

Nonionic surfactants that may be used include the primary and secondary alcohol ethoxylates, especially the C₈ to C₂₀ aliphatic alcohols ethoxylated with an average of from 1 to 20 moles of ethylene oxide per mole of alcohol, and more especially the C₁₀ to C₁₅ primary and secondary aliphatic alcohols ethoxylated with an average of from 1 to 10 moles of ethylene oxide per mole of alcohol. Non ethoxylated nonionic surfactants include alkylpolyglycosides, glycerol monoethers, and polyhydroxyamides (glucamide). It is preferred if the level of non-ionic surfactant is from 0 wt % to 30 wt %, preferably from 1 wt % to 25 wt %, most preferably from 2 wt % to 15 wt %, by weight of a fully formulated composition comprising the microcapsules of the invention.

Suitable amphoteric surfactants preferably are betaine surfactants. Examples of suitable amphoteric surfactants include, but are not limited to, alkyl betaines, alkylamido betaines, alkyl sulfobetaines, alkyl sultaines and alkylamido sultaines; preferably, those having 8 to about 18 carbons in the alkyl and acyl group. It is preferred that the amount of the amphoteric surfactant is 0 to 20 wt %, more preferably from 1 to 10 wt %, by weight of the composition.

It is also possible to include certain mono-alkyl cationic surfactants. Cationic surfactants that may be used include quaternary ammonium salts of the general formula R¹R²R³R⁴N⁺X⁻ wherein the R groups are long or short hydrocarbon chains, typically alkyl, hydroxyalkyl or ethoxylated alkyl groups, and X is a counter-ion (for example, compounds in which R¹ is a C₈-C₂₂ alkyl group, preferably a C₈-C₁₀ or C₁₂-C₁₄ alkyl group, R² is a methyl group, and R³ and R⁴, which may be the same or different, are methyl or hydroxyethyl groups); and cationic esters (for example, choline esters).

Water-soluble skin benefit agents may optionally be formulated into the compositions of the invention. A variety of water-soluble skin benefit agents can be used and the level can be from 0.1 to 50% but preferably from 1 to 30% by weight of the composition. These materials include, but are not limited to, polyhydroxy alcohols. Preferred water-soluble skin benefit agents are glycerin, sorbitol and polyethylene glycol.

Water-insoluble skin benefit agents may also be formulated into the compositions as conditioners and moisturizers. Examples include silicone oils; hydrocarbons such as liquid paraffins, petrolatum, microcrystalline wax, and mineral oil; and vegetable triglycerides such as sunflower seed and cottonseed oils.

Some compositions may include thickeners. These may be selected from cellulosics, natural gums and acrylic polymers but not limited by this thickening agent types. Among the cellulosics are sodium carboxymethyl cellulose, hydroxyethyl cellulose, hydroxypropyl methylcellulose and combinations thereof. Suitable gums include xanthan, pectin, karaya, agar, alginate gums and combinations thereof. Among the acrylic thickeners are homopolymers and copolymers of acrylic and methacrylic acids including carbomers such as Carbopol 1382, Carbopol 982, Ultrez, Aqua SF-1 and Aqua SF-2 available from the Lubrizol Corporation. Amounts of thickener may range from 0.01 to 3% by weight of the active polymer (outside of solvent or water) in the compositions.

Preservatives can desirably be incorporated into the compositions of this invention to protect against the growth of potentially harmful microorganisms. Particularly preferred preservatives are phenoxyethanol, methyl paraben, propyl paraben, imidazolidinyl urea, sodium dehydroacetate and benzyl alcohol. The preservatives should be selected having regard for the use of the composition and possible incompatibilities between the preservatives and other ingredients. Preservatives are preferably employed in amounts ranging from 0.01% to 2% by weight of the composition.

A variety of other optional materials may be formulated into the compositions. These may include: antimicrobials such as 2-hydroxy-4,2′,4′-trichlorodiphenylether (triclosan), 2,6-dimethyl-4-hydroxychlorobenzene, and 3,4,4′-trichlorocarbanilide; scrub and exfoliating particles such as polyethylene and silica or alumina; cooling agents such as menthol; skin calming agents such as aloe vera; and colorants.

In addition, the compositions of the invention may further include 0.5 to 10% by weight of sequestering agents, such as tetra sodium ethylenediaminetetraacetate (EDTA), EHDP or mixtures; opacifiers and pearlizers such as ethylene glycol distearate, titanium dioxide or Lytron 621 (Styrene/Acrylate copolymer); all of which are useful in enhancing the appearance or properties of the product.

Preferably the composition comprises water in an amount of at least 5% by weight of the composition, more preferably at least 25%, even more preferably 40 to 90% and still even more preferably at least 60 to 85% by weight of the composition.

Preferably, the composition has a viscosity of at least 5 mPa·s, more preferably in the range 8 to 10000 mPa·s, even more preferably 10 to 1000 mPa·s, and most preferably 20 to 200 mPa·s, when measured at 20 degrees C. at a relatively high shear rate of about 20 s⁻¹. Preferably, the composition is in the form of fluid.

The cosmetic composition refers to a composition suitable for topical application to human skin, preferably is a rinse-off product. The term “rinse-off” as used with reference to compositions herein means a composition that is applied to or rubbed on the skin, and then washed off. The term “skin” as used herein includes the skin on the face (except eye lids and lips), neck, chest, abdomen, back, arms, under arms, hands, and legs. Preferably “skin” means includes the skin on the face (except eye lids and lips) and under arms, more preferably skin means skin on the face other than lips and eyelids.

The present invention also provided use of a combination of component (a) selected from C₈ to C₁₅ fatty acid and salt thereof; and component (b) selected from C₂ to C₆ hydroxy carboxylic acid and salt thereof, wherein the weight ratio of total component (a) to total component (b) is at least 3:1, for use as anti-acne or anti-bacterial agent, preferably in cosmetic composition.

Preferably, the combination is a combination of component (a) selected from C₁₂ to C₁₄ fatty acid and salt thereof, and component (b) selected from C₂ to C₆ hydroxy carboxylic acid and salt thereof, with a weight ratio of total component (a) to total component (b) being at least 3:1.

Preferably, the combination is a combination of component (a) selected from C₁₂ to C₁₄ fatty acid and salt thereof, and component (b) selected from C₂ to C₄ alpha hydroxy carboxylic acid and salt thereof, with a weight ratio of total component (a) to total component (b) being at least 3:1.

Preferably, the combination is a combination of component (a) selected from lauric acid, myristic acid, salt thereof, and combination thereof, and component (b) selected from C₂ to C₄ alpha hydroxy carboxylic acid and salt thereof, with a weight ratio of total component (a) to total component (b) being at least 3:1.

Preferably, the combination is a combination of component (a) selected from lauric acid, myristic acid, salt thereof, and combination thereof, and component (b) selected from lactic acid and salt of lactic acid, with a weight ratio of total component (a) to total component (b) being at least 3:1.

Preferably, the combination is a combination of component (a): lauric acid, and component (b): lactic acid, with a weight ratio of total component (a) to total component (b) being at least 3:1.

For sake of clarity, the combination of the present invention typically provides a greater anti-bacterial, particularly anti-acne benefit in comparison to component (a), component (b) or a combination of component (a) and component (b) with a weight ratio of total component (a) to total component (b) being less than 3:1. Preferably the combination is present in cosmetic composition.

The present invention also provides a cosmetic composition comprising a combination of component (a) selected from C₈ to C₁₅ fatty acid and salt thereof; and component (b) selected from C₂ to C₆ hydroxy carboxylic acid and salt thereof, with the weight ratio of total component (a) to total component (b) being at least 3:1, for providing anti-acne or anti-bacterial benefit.

The cosmetic composition of the present invention typically provides a greater anti-acne and anti-bacterial benefit in comparison to a cosmetic composition that does not contain component (a) and component (b) with a weight ratio of total component (a) to total component (b) being at least 3:1.

The present invention also provides use of a combination of component (a) selected from C₈ to C₁₅ fatty acid and salt thereof; and component (b) selected from C₂ to C₆ hydroxy carboxylic acid and salt thereof, with the weight ratio of total component (a) to total component (b) being at least 3:1 in the manufacture of medicants for providing anti-acne, or anti-bacterial function.

The present invention also provides use of a combination of component (a) selected from C₈ to C₁₅ fatty acid and salt thereof; and component (b) selected from C₂ to C₆ hydroxy carboxylic acid and salt thereof, with the weight ratio of total component (a) to total component (b) being at least 3:1, as anti-acne or anti-bacterial agent.

The present invention also provides use of a combination of component (a) selected from C₈ to C₁₅ fatty acid and salt thereof; and component (b) selected from C₂ to C₆ hydroxy carboxylic acid and salt thereof, with the weight ratio of total component (a) to total component (b) being at least 3:1 in a method for providing anti-acne or anti-bacterial benefit, the method comprising topical application of a cosmetic composition comprising said combination.

Preferably the use is non-therapeutic. Preferably the method is non-therapeutic. Preferably the anti-acne or anti-bacterial benefit is synergistic anti-acne or anti-bacterial benefit. The term non-therapeutic typically means for cosmetic purposes and not curative or therapeutic purposes.

The following examples are provided to facilitate an understanding of the invention. The examples are not intended to limit the scope of the claims.

### EXAMPLES

### Example 1

This example demonstrates the synergy on anti-acne between lauric acid and lactic acid.

TABLE 1

| | Sample (g) | | | | | |
|---|---|---|---|---|---|---|
| | A | B | C | D | E | 1 |
| Lauric acid | 1.00 | — | 0.167 | 0.50 | 0.667 | 0.833 |
| Lactic acid | — | 1.00 | 0.833 | 0.50 | 0.333 | 0.167 |
| Ethanol | 9.00 | 9.00 | 9.00 | 9.00 | 9.00 | 9.00 |
| ZOI (mm) | 27.4 | 8.0 | 14.9 | 11.3 | 27.7 | 33.6* |

*Significantly better than any of A, B, C, D, E (p < 0.05)

Zone of Inhibition (ZOI) of different samples toward Propionibacterium acne (P. acnes) was tested. Samples was prepared according to Table 1. 20 μL of each sample or control sample (aseptic distilled water) were dipped onto identical filter papers and all of these filter papers were dried. Cultures of Propionibacterium acnes were washed and diluted by reinforced clostridial medium (RCM) to suspension with concentration of 1×10⁸ to 9×10⁸ cfu/mL. The suspension was evenly smeared on a nutrient agar culture medium plate by spiral spread. The dried filter papers with samples in Table 1 were closely applied onto the plated with sufficient distances. The plate was covered and cultured at 37° C. for 70 to 72 hours. The samples diffuse into the agar are assayed for an ability to produce a zone of inhibition. The diameters of ZOI for different samples were recorded in Table 1. The bigger the ZOI is, the better anti-acne efficiency the sample provides. The last row shows the test results.

It is evident from Table 1 that Sample 1 provided a significant better anti-acne efficiency that any of sample A, B, C, D, and E. It was surprisingly found that it is capable of providing a synergistic anti-acne benefit when the weight ratio of the lauric acid to the lactic acid is at least 3:1.

### Example 2

This example demonstrates the synergy on anti-acne between myristic acid and lactic acid.

TABLE 2

Sample (g)

| | F | G | H | I | 2 |
|---|---|---|---|---|---|
| Myristic acid | 2.50 | — | 0.714 | 1.25 | 2.083 |
| Lactic acid | — | 2.50 | 1.786 | 1.25 | 0.417 |
| Ethanol | 7.50 | 7.50 | 7.50 | 7.50 | 7.50 |
| ZOI (mm) | 9.7 | 15.7 | 12.0 | 11.2 | 17.72* |

*Significantly better than any of F, G, H, I (p < 0.05)

The experiments were conducted in a similar manner as described in Example 1 except that samples in Table 2 were prepared and employed. The last row of Table 2 shows the results.

It is evident from Table 1 that sample 2 provided a significant better anti-acne efficiency that any of sample F, G, H, and I. It was surprisingly found that it is capable of providing a synergistic anti-acne benefit when the weight ratio of the myristic acid to the lactic acid is at least 3:1.

### Example 3

This example demonstrates there is no synergy on anti-acne between palmitic acid and lactic acid in below samples.

TABLE 3

Sample (g)

| | J | K | L | M | N |
|---|---|---|---|---|---|
| Palmitic acid | 2.50 | — | 0.714 | 1.25 | 2.083 |
| Lactic acid | — | 2.50 | 1.786 | 1.25 | 0.417 |
| Ethanol | 7.50 | 7.50 | 7.50 | 7.50 | 7.50 |
| ZOI (mm) | 8.9 | 15.7 | 8.9 | 12.2 | 10.7 |

The experiments were conducted in a similar manner as described in Example 1 except that samples in Table 3 were prepared and employed in the test. The last row of Table 3 shows the results.

As shown in Table 3, there is no synergistic anti-acne efficiency when combining palmitic acid and lactic acid.

### Example 4

This example demonstrates a cometic composition containing the combination of lauric acid and lactic acid.

A skin cleansing composition was prepared according to Table 4 by following standard procedure.

TABLE 4

| | Samples (wt %*) |
|---|---|
| Ingredient | 1 |
| Water | To 100 |
| Sodium Laureth Sulfate | 9.00 |
| Cocamidopropyl Betaine | 1.59 |
| Glycerin | 3.00 |
| Propylene Glycol | 1.50 |
| Tetrasodium EDTA | 0.05 |
| Lauric acid | 0.833 |
| Lactic acid | 0.167 |
| Preservative | 0.55 |
| Fragrance | 0.56 |

1. A cosmetic composition comprising:
component (a) selected from C₈ to C₁₅ fatty acid and salt thereof; and
component (b) selected from C₂ to C₆ hydroxy carboxylic acid and salt thereof,
wherein the weight ratio of total component (a) to total component (b) is at least 3:1, the fatty acid is C₁₀ to C₁₄ fatty acid and the hydroxy carboxylic acid is C₂ to C₄ alpha hydroxy carboxylic acid,
wherein the cosmetic composition further comprises a surfactant which comprises soap, non-soap anionic, cationic, non-ionic, amphoteric surfactant and mixtures thereof.

2. The composition according to claim 1 wherein the fatty acid is lauric acid, myristic acid, or a combination thereof and the composition further comprises at least 5% by weight water.

3. The composition according to claim 1 wherein the fatty acid is saturated fatty acid without substitution, preferably C₁₀ to C₁₄ saturated fatty acid without substitution.

4. The composition according to claim 1 wherein total amount of component (a) is 0.05 to 5% by weight of the composition.

5. The composition according to claim 1 wherein the total amount of component (a) is 0.1 to 18% by weight of the composition.

6. The composition according to claim 1 wherein the C₂ to C₆ hydroxy carboxylic acid is lactic acid.

7. The composition according to claim 1 wherein the total amount of component (b) is 0.02 to 2% by weight of the composition.

8. The composition according to claim 1 wherein total weight of component (a) to total weight of component (b) are at a weight ratio from 3:1 to 30:1.

9. The composition according to claim 1 wherein the component (a) is selected from C₁₂ to C₁₄ fatty acid and salt thereof, and component (b) is selected from C₂ to C₄ alpha hydroxy carboxylic acid and salt thereof and component (a) and component (b) are at a weight ratio from 3.5:1 to 8.5:1.

10. The composition according to claim 9 wherein the weight ratio of total weight of C₁₂ to C₁₄ fatty acid salt thereof to the total weight of C₂ to C₄ alpha hydroxy carboxylic acid and salt thereof is 4:1 to 7:1.

11. The composition according to claim 1 wherein the component (a) is lauric acid, and component (b) is lactic acid.

12. The composition according to claim 10 wherein the composition does not comprise soap.

13. The composition according to claim 1 wherein the composition provides an anti-acne or anti-bacterial benefit.

14. The composition according to claim 13 wherein the composition does comprise soap.

15. (canceled)

16. The composition according to claim 1 wherein the composition comprises amphoteric surfactant and anionic surfactant.
